(19) United States
(12) Patent Application Publication
(10) Pub. No.: US 2022/0175900 A1
(43) Pub. Date: Jun. 9, 2022

(54) METHOD FOR PREPARATION OF CANCER/TESTIS ANTIGEN-SPECIFIC T-CELLS

(71) Applicant: Cytovac A/S, Hørsholm (DK)

(72) Inventors: Alexei Kirkine, Copenhagen Ø (DK); Karine Dzhanzhugazyan, Copenhagen Ø (DK)

(73) Assignee: Cytovac A/S, Hørsholm (DK)

(21) Appl. No.: 17/602,917

(22) PCT Filed: Apr. 8, 2020
(86) PCT No.: PCT/EP2020/059962

§ 371 Date: Oct. 11, 2021

(30) Foreign Application Priority Data
Apr. 12, 2019 (EP) 19168957.9

(51) Int. Cl. A61K 39/00; A61P 35/00; C12N 5/0783

(52) U.S. Cl. CPC A61K 39/001184; A61P 35/00; C12N 5/0638; C12N 5/0646; A61K 2039/5158; A61K 2039/892; C12N 2502/1121; C12N 2501/06; C12N 2501/22; C12N 2501/2301; C12N 2501/2302; C12N 2501/2304; C12N 2501/25; C12N 2501/02

(57) ABSTRACT

Provided is a method for preparation of a composition comprising activated human CD8⁺ and natural killer (NK) lymphocytes. The method entails use of mature dendritic cells as feeder cells added at an early stage in CD4⁺ mediated activation of the CD8⁺ cells. Also provided is a method for treatment of cancer using the cells obtained from the process.

### FIELD OF THE INVENTION

The present invention relates to methods for producing effector cells that are useful in adoptive immunotherapy and also relates to the field of adoptive immunotherapy.

### BACKGROUND OF THE INVENTION

Among different approaches to cancer immunotherapy, adoptive immunotherapy shows high efficiency in inducing tumour regression in selected malignancies. Two major types of the adoptive immunotherapy are currently the most successful: CAR-T therapy and TIL therapy.

In the CAR-T approach, T lymphocytes are transfected with Chimeric Antigen Receptor (CAR), a fusion protein between an Fab antibody fragment and a fragment of a T cell receptor. This approach demonstrates high efficiency in treatment of B cell malignancies, while it is much less effective for treatment of solid tumours.

Another approach, based on the employment of lymphocytes expanded from autologous tumour (tumour infiltrating lymphocytes, TIL), demonstrates high clinical efficiency in melanoma patients. In a number of clinical trials employing TILs, it has been shown that the clinical effect correlate with number of the injected cells, rate of their proliferation and their lytic activity against autologous tumour cells. Unfortunately, isolation and expansion of tumour infiltrating lymphocytes with cytolytic activity against autologous tumours from other types of malignancies are significantly more difficult, restricting broad application of this technology.

The present inventors have recently developed a new method of adoptive immunotherapy of cancer, based on generation of cytotoxic lymphocytes specifically targeting a broad spectrum of cancer-testis antigens, which constitutes a group of shared tumour antigens appearing in tumour cells as a result of genome wide DNA de-methylation (Kirkin et al., 2018; WO 2008/081035). This procedure consists of four steps (see FIG. 1):
1) generation of monocyte derived mature dendritic cells;
2) Induction of proliferation of predominantly CD4+ cells after co-culture of the mature dendritic cells and peripheral blood lymphocytes (PBLs);
3) Treatment of the activated CD4-enriched lymphocytes with a DNA de-methylating agent leading to induction of expression of a broad spectrum of cancer/testis antigens; and
4) Generation of cytotoxic lymphocytes (an “immunization step”) after co-culture of DNA de-methylated activated CD4⁺ enriched lymphocytes with unstimulated PBLs.

This method is universal, and can be applied to treatment of many types of malignancies.

The present inventors have already demonstrated its efficiency in treatment of patients with relapsed glioblastoma (Kirkin et al., 2018). Here, 3 out of 10 patients who received the planned 3 injections of the therapeutic cells exhibited tumour regression.

The lack of the response in some patients may be related to insufficient numbers of the injected cells. Therefore, further improvement of this technology leading to generation of significant larger number of cells is of interest.

Currently, the main procedure used to upscale the production of cytotoxic lymphocytes is the so-called “rapid expansion protocol” (Dudley et al., 2003). The principle element of this procedure is use of feeder cells, consisting of mixture of irradiated allogeneic PBMCs from several healthy donors. There are, however, several disadvantages of using of such feeder cells prepared according to the rapid expansion protocol for expansion of cytotoxic lymphocytes. Main issues are: a) the allogeneic nature of feeder cells can lead to unwanted reactivity to the alloantigens present on feeder cells, and therefore decrease the proportion of the lymphocytes specific against tumour antigens; and b) the need of irradiation significantly decreases the wide use of this protocol due to presence of the necessary irradiation equipment only in the specialized centres.

There is hence a need for an improved alternative to the method disclosed in Dudley et al., 2003 for upscaling cytotoxic lymphocyte production.

### OBJECT OF THE INVENTION

It is an object of embodiments of the invention to provide an improved method for producing activated cytotoxic lymphocytes. It is a further object to provide improved methods of adoptive immunotherapy that utilise activated cytotoxic lymphocytes.

### SUMMARY OF THE INVENTION

The present inventors have employed a novel approach in order to increase the final number of effector cells in a process such as the one set forth above in steps 1-4. It has been found that addition—as feeder cells—of autologous mature dendritic cells already at the intermediate step of the procedure described above in steps 1-4, i.e. either at the start of treatment with a DNA de-methylating agent, or at the initial period of co-incubation of de-methylated T-helper cells with unstimulated peripheral blood lymphocytes, provides for a significant increase of the yield of effector cells. Hence, the present invention employs the novel finding that the same mature autologous DCs are useful for both the process of inducing proliferation of CD4⁺ cells and for stimulating at an early stage proliferation of CD8⁺ cells that are being activated in the presence of CD4⁺ cells.

It is in this connection important that the procedure is about 1 week faster than what has been disclosed in WO 2008/081035 where the same type of feeder cells, i.e. autologous mature dendritic cells were added on day 11 after initiation of co-culture of de-methylated T-helper cells and unstimulated lymphocytes, and where treatment with lymphocytes could not be instigated earlier than on day 17 or 18 after initiation of the co-culture. In the case of treatment of cancers, time is an important factor, and the early start of treatment naturally provides for better chances of a positive clinical outcome. It is also important that the final product (the effector cells) has the same relative lytic activity, phenotype, and in some experiments the same or higher ability to produce GM-CSF has been observed. Noteworthy, in some experiments when adding DCs at a very early stage, the proliferative activity of the final effector cells has proven to be higher compared to the existing standard procedure.

So, in a 1^{st} aspect the present invention relates to a method for preparation of a composition comprising activated human CD8⁺ and natural killer (NK) lymphocytes, comprising
1) isolating mononuclear cells from a blood sample from a human donor and separating the PBMCs into a fraction enriched for monocytes and a fraction enriched for lymphocytes;
2) culturing a portion of the monocyte-enriched fraction under conditions that facilitate maturation of dendritic cells;
3) subsequently mixing a first portion of the mature dendritic cells obtained in step 2 with a first portion of the lymphocyte fraction obtained in step 1);
4) co-culturing the mixed cells obtained in step 3 to stimulate proliferation of CD4+ lymphocytes, thereby increasing the CD4⁺/CD8⁺ ratio compared to the lymphocytes obtained from step 1;
5) isolating proliferating lymphocytes from the co-cultured cells of step 4, and subsequently contacting them with an agent that induces expression of cancer/testis antigens followed by a period of culture that results in said expression of cancer/testis antigens;
6) mixing the cancer/testis antigen expressing lymphocytes obtained in step 5 with a second portion of the fraction enriched for lymphocytes in step 1; and
7) subsequently culturing the lymphocyte mixture from step 6 to stimulate proliferation of CD8+ and NK lymphocytes, wherein a second portion of mature dendritic cells obtained from step 2 are added to the proliferating lymphocytes in any of steps 5-7 and at the latest 6 days after step 6.

In a 2^{nd} aspect of the invention is provided a method for preparation of a composition comprising activated human CD8+ and natural killer (NK) lymphocytes, comprising the subsequent steps of
a) contacting a first composition of human cells comprising proliferating CD4⁺ lymphocytes with an agent that induces expression of cancer/testis antigens followed by a culturing period that results in said expression of cancer/testis antigens by cells in the first composition; and
b) adding a second composition of human cells comprising unstimulated peripheral blood lymphocytes to the first composition of cells and culturing the combined compositions of cells to stimulate proliferation of CD8+ and NK lymphocytes;

wherein a third composition of human cells comprising mature dendritic cells is added in step a or b and at the latest 6 days after initiation of step b, and wherein the first, second and third compositions of human cells are isogeneic.

In a 3^{rd} aspect, the present invention provides a method for preparation of a composition comprising activated human CD8+ and natural killer (NK) lymphocytes, comprising mixing
a first composition of human cells comprising cancer/testis antigen expressing CD4+ lymphocytes with
a second composition of human cells comprising unstimulated peripheral blood lymphocytes

and culturing the combined compositions of cells to stimulate proliferation of CD8+ and NK lymphocytes;

wherein a third composition of human cells comprising mature dendritic cells is added to the combined compositions at the latest 6 days after mixing the first and second composition and wherein the first, second and third compositions of cells are isogeneic.

In a 4^{th} aspect, the present invention provides a method for treatment of cancer in a patient, comprising administering a composition of cells prepared according to the method of any of aspects 1-3 of the invention.

In a fifth aspect, the present invention provides a composition of cells prepared according to any of aspects 1-3 for use in therapy, notably for use in a method according to the 4^{th} aspect of the invention.

Finally, in a 6^{th} aspect, the present invention relates generally to use of mature dendritic cells as feeder cells in a culture of a mixed cell composition initially comprising cancer/testis antigen expressing CD4+ lymphocytes and unstimulated peripheral blood lymphocytes, where the mature dendritic cells are added to the mixed cell composition no later than 6 days after initially establishing the mixed cell composition.

### LEGENDS TO THE FIGURE

FIG. 1: Overview of general production process.

Vertical lines separate phases I, II, III, and IV, where phase I (up to day 6) is the generation of mature dendritic cells, phase II (days 6-13) is the generation of CD4⁺-enriched lymphocytes, phase III (days 13-15) is induction of cancer/testis antigens (CTA) by the proliferating lymphocytes, and phase IV (days 15-26) is the immunization step, where CD8⁺ cells and NK cells are stimulated by the CD4⁺ CTA expressing cells. “Mon” denotes monocytes, “Lym” denotes lymphocytes.

FIG. 2: Bar graph showing numbers of cells from 6 donors after culture with dendritic cells added at day 15 or 17 or not added.

Cell counts at day 26 are shown for 6 donors (termed 38/17, AK-19, 42/17, 43/17, 44/17, and 48/17) where cells are treated according to the prior art method (left bar for each donor), the inventive method with addition of dendritic cells at day 15 (middle bar for each donor), and with addition of dendritic cells at day 17 (right bar for each donor).

FIG. 3: Bar graph showing lytic activity of T lymphocytes from 6 donors after culture with dendritic cells added at day 15.

Activity data at day 26 is shown for 6 donors (termed 38/17, AK-19, 42/17, 43/17, 44/17, and 48/17) where cells are treated according to the prior art method (left bar for each donor), the inventive method with addition of dendritic cells at day 15 (middle bar for each donor), and with addition of dendritic cells at day 17 (right bar for each donor).

FIG. 4: Bar graph showing numbers of cells from 14 donors after culture with dendritic cells added at day 15.

Cell counts at day 26 from 14 donors (38/17, AK-19, 42/17, 43/17, 44/17, 48/17, DO, LI, 52/17d7, 54/17d7, 55/17d7, and AK-18) after culture according to the prior art method (left bar for each donor) and the method of the invention in the embodiment where dendritic cells are added at day 17. Numbers show the ratio between cell numbers obtained with the 2 methods, with an average increase of cell numbers of 6.9±3.6 times.

FIG. 5: Bar graph showing percentage of CD3⁺ cells from 14 donors after culture with dendritic cells added at day 17.

Proportion of CD3⁺ cells at day 26 in the cell preparations obtained after culture of blood from the same donors as in FIG. 4 according to the prior art method (left bar for each donor) and the method of the invention in the embodiment where dendritic cells are added at day 17.

FIG. 6: Bar graph showing percentage of NK cells (CD56⁺/CD3⁻) from 14 donors after culture with dendritic cells added at day 17.

Proportion of NK cells at day 26 in the cell preparations obtained after culture of blood from the same donors as in FIG. 4 according to the prior art method (left bar for each donor) and the method of the invention in the embodiment where dendritic cells are added at day 17.

FIG. 7: Bar graph showing percentage of CD62L expressing T cells from 14 donors after culture with dendritic cells added at day 17.

Proportion of CD62L⁺ T cells at day 26 in the cell preparations obtained after culture of blood from the same donors as in FIG. 4 according to the prior art method (left bar for each donor) and the method of the invention in the embodiment where dendritic cells are added at day 17.

FIG. 8: Bar graph showing percentage of CD62L expressing NK cells from 14 donors after culture with dendritic cells added at day 15.

Proportion of CD62L⁺ NK cells at day 26 in the cell preparations obtained after culture of blood from the same donors as in FIG. 4 according to the prior art method (left bar for each donor) and the method of the invention in the embodiment where dendritic cells are added at day 17.

FIG. 9: Bar graph showing final number of cells from 4 donors after culture with dendritic cells added at day 13.

Cell counts at day 26 are shown for 4 donors (termed 116/17, 117/17, 118/17, and 119/17) where cells are treated according to the prior art method (left bar for each donor) and the inventive method with addition of dendritic cells at day 13 (right bar for each donor).

FIG. 10: Bar graphs showing relative cytotoxic activity of cells from 4 donors after culture with dendritic cells added at day 13.

Exemplary cell counts at day 26 are shown for 4 donors (termed 116/17, 117/17, 118/17, and 119/17) where cells are treated according to the prior art method (left bar for each donor) and the inventive method with addition of dendritic cells at day 13 (right bar for each donor).

FIG. 11: Bar graph showing final number of cells from 12 donors after culture with dendritic cells added at day 13.

Cell counts at day 26 are shown for 12 donors (termed 40/18, 41/18, 43/18, 44/18, 45/18, 46/18, 47/18, 48/18, 49/18, 50/18, 55/18, and 56/18) where cells are treated according to the prior art method (left bar for each donor) and the inventive method with addition of dendritic cells at day 13 (right bar for each donor).

FIG. 12: Bar graph showing proliferative activity of cells from 8 donors after culture with dendritic cells added at day 13.

Final concentrations of cells harvested on day 26 and subsequently cultured for 3-4 days in the presence of IL-2. Data is shown for cells from 8 donors (termed 40/18, 41/18, 43/18, 44/18, 45/18, 46/18, 47/18, and 48/18), where cells are treated according to the prior art method (left bar for each donor) and the inventive method with addition of dendritic cells at day 13 (right bar for each donor).

FIG. 13: Bar graph showing final number of cells from 5 patients with glioblastoma multiforme after culture with dendritic cells added at day 13.

Cell counts at day 26 are shown for 5 patients (pt01007, pt01014, pt01011, pt03007, and two cultures of patient pt04006: pt04006-1, and pt04006-9), where cells are treated according to the prior art method (left, almost invisible, bar for each patient) and the inventive method with addition of dendritic cells at day 13 (right bar for each patient). Numbers of cells (×10⁶) are also indicated in the table below the graph.

### DETAILED DISCLOSURE OF THE INVENTION

### Definitions

“Cancer/testis antigens” (CTAs) is a group of antigens, which are expressed by a broad spectrum of cancers. The group includes such antigens as MAGE (including MAGE-1, MAGE-2, and MAGE-3), BAGE, GAGE, NY-ESO-1 and BORIS, which are all cancer-associated antigens that can be safely targeted, since they are not normally expressed in healthy cells in vital tissues. Previously it has been demonstrated that the expression by cancer cells of CTAs is a consequence of genome-wide de-methylation (including promoter de-methylation at CpG regions), which occurs in many cancers.

“Mononuclear cells” (also term “peripheral blood mononuclear cells”, abbreviated PBMC) denotes any cells of peripheral blood that have a rounded nucleus. The two main types of mononuclear cells are lymphocytes and monocytes, of which the latter have the ability to differentiate into macrophages and dendritic cells.

“Mature dendritic cells” (mature DCs) are in the present context dendritic cells that are obtainable by culturing monocytes under conditions described herein and which in contrast to immature dendritic cells a high potential for T-cell activation. These mature dendritic cells, which are obtained by plating and culturing adhering monocytes, subsequently treating with IL-4 (and/or IL-13) and GM-CSF to differentiate the monocytes into immature DCs and thereafter treating the immature DCs with TNF-alpha, IL-1beta, IL-6, and prostaglandin E2, are not loaded with antigen as would be the case for mature DCs isolated from lymphoid tissue.

“CD4⁺ lymphocytes” or “CD4⁺ cells” (the terms are used interchangeably herein) refer to lymphocytes of the T-helper subset. Among their functions are stimulation of B-cells and they also play an important role in the activation of CD8⁺ lymphocytes.

“CD8⁺ lymphocytes” or “CD8⁺ cells” or “cytotoxic T cells” (the terms are used interchangeably herein) refer to antigen specific lymphocytes that are capable of recognizing and killing cells that display MCH class I restricted T-cell epitopes.

“Natural killer cells” or “NK cells” or “NK lymphocytes” are antigen unspecific lymphocytes, which form part of the fast-reacting innate immune system, and which, as is the case of cytotoxic T cells, have the ability to kill cells. This occurs as part of recognition of stress-induced proteins characteristic for cancer cells. NK cells have a preferential ability to target cells that do not express MHC class I molecules.

The expression “increasing the CD4+/CD8+ ratio” is in the present context meant to indicate that a lymphocyte population that has been co-cultured with mature DCs as taught herein provides for a preferential expansion of the CD4⁺ subset of lymphocytes. It has been demonstrated that such co-culture, which forms part of the technology disclosed in WO 2008/081035, provides for a significant increase in CD4⁺ cells compared to CD8⁺ cells.

“An agent that induces expression of cancer/testis antigens” denotes a substance or composition, which is able to produce in a treated cell an effect corresponding to what has been observed in many cancers, namely that CTAs are expressed due to genome-wide changes. Typically, substances that can cause DNA to de-methylate are useful; good examples are 5-aza-2′-deoxycytidine, 5-azacytldine, 5-fluoro-2′-deoxycytidine, guadecitabine, and zebularine. Of these, the preferred de-methylation agent is 5-aza-2′-deoxycytidine (also termed 5-Aza-CdR or simply AzaC herein), which is a cytidine analogue that acts as a nucleic acid synthesis inhibitor. This substance under the name decitabine (marketed under the tradename DACOGEN®) acts via inhibition of DNA methyltransferase. As a viable alternative to use of de-methylating agent can be mentioned use of agents that induce the CTAs by means of histone acetylation an example of such an agent is the histone deacetylase inhibitor trichostatin A.

### Specific Embodiments of the Invention

1^{st} Aspect of the Invention

The 1^{st} aspect of the invention relates as indicated above—to a method for preparation of a composition comprising activated human CD8⁺ and natural killer (NK) lymphocytes, comprising
1) isolating mononuclear cells from a blood sample from a human donor and separating the PBMCs into a fraction enriched for monocytes and a fraction enriched for lymphocytes;
2) culturing a portion of the monocyte-enriched fraction under conditions that facilitate maturation of dendritic cells;

3) subsequently mixing a first portion of the mature dendritic cells obtained in step 2 with a first portion of the lymphocyte fraction obtained in step 1;
4) co-culturing the mixed cells obtained in step 3 to stimulate proliferation of CD4+ lymphocytes, thereby increasing the CD4⁺/CD8⁺ ratio compared to the lymphocytes obtained from step 1;
5) isolating proliferating lymphocytes from the co-cultured cells of step 4, and subsequently contacting them with an agent that induces expression of cancer/testis antigens followed by a period of culture that results in said expression of cancer/testis antigens;
6) mixing the cancer/testis antigen expressing lymphocytes obtained in step 5 with a second portion of the fraction enriched for lymphocytes in step 1; and
7) subsequently culturing the lymphocyte mixture from step 6 to stimulate proliferation of CD8+ and NK lymphocytes,

wherein a second portion of mature dendritic cells obtained from step 2 are added to the proliferating lymphocytes in any of steps 5-7 and at the latest 6 days after step 6.

It will hence be clear that all cells (including lymphocytes and monocytes/dendritic cells) are derived from the same donor and hence are isogeneic of origin. The method is particularly useful for preparation of cells for use in personalised adoptive immunotherapy, where a patient's own lymphocytes are activated so as to be powerful effector cells with the ability to target/kill tumour cells that express cancer/testis antigens.

The early addition of the mature dendritic cells after induction of the expression of cancer/testis antigens is a hallmark of the present invention. In the prior art, the addition of such isogeneic feeder cells has taken place at a much later phase, i.e. after the co-culture of the cancer/testis antigen expressing cells and the lymphocytes isolated in step 1, but as evidenced herein, the application of the isogeneic feeder cells as early as in step 5 (i.e. typically 13 days from the 1^{st} step) provides for significant increases in yields of effector cells at the end of step 7. It will hence be understood that the mature DCs can be added at any time-point in the procedure from the time of start of induction of the CTAs until at most 6 days after step 6.

This means that the mature DCs may be added on any of days 0, 1, 2, 3, 4, 5, and 6 after step 6, but also prior to that, i.e. 1, 2, or more days prior to step 6, but not prior to step 5; in the first case, the second portion of mature dendritic cells may be added in step 6 concurrently with or after mixing the cancer/testis antigen expressing lymphocytes with second portion of the fraction enriched for lymphocytes and in the latter case, the second portion of mature dendritic cells may be added in step 5 prior to, concurrently with or after contacting the proliferating lymphocytes with the agent that induces expression of cancer/testis antigens.

According to a preferred embodiment, steps 1-2 together last about 6 days, steps 3-4 together last about 7 days, step 5 lasts 2 days, and steps 6-8 together last about 11 days.

Therefore, it is in accordance with the above preferred that the second portion of mature dendritic cells is added 13-17 days after commencement of step 2, preferably 15-17 days after commencement of step 2.

Step 1 consists of a step of separation of monocytes from lymphocytes after provision of a sample of PBMCs. After this separation, both the lymphocyte fraction and the monocyte fractions are divided into at least 2 portions each. Since cells from the lymphocyte fraction are not entering the process described above until after step 2, and since further cells from the lymphocyte fraction are not entering the process until after step 5, the at least 2 portions of the fraction enriched for lymphocytes is frozen, one between steps 1 and 2, and another between steps 1 and 6. Likewise, the second portion of the mature dendritic cells is kept frozen between step 2 and the addition of this portion in step 5 or step 6. Under normal circumstances, the first portion of the mature DCs is used directly after step 1, i.e. without being frozen.

Step 2 is essentially carried out according to known methods for preparing mature DCs from monocytes in culture; these known methods include addition, during the course of culture, of granulocyte-macrophage colony stimulating factor (GM-CSF) and Interleukin 4 (IL-4) (and/or Interleukin 13) to obtain immature DCs, followed by addition of TNFα to obtain the mature DCs. Additionally, Interleukin 1β (IL-1β), Interleukin 6 (IL-6), and prostaglandin E2 (PGE2) can advantageously be added in the phase of preparing the mature DCs.

Steps 4-7 are generally carried out as disclosed in WO 2008/081035 with the exception of the early addition of mature DC feeder cells in steps 5-7, which is disclosed herein. Hence, any generic disclosure relating to these steps also apply to the present process, and the processes described below for the 2^{nd} and 3^{rd} aspect of the invention.

2^{nd} Aspect of the Invention

This aspect of the invention relates as does the 1^{st} (and 3^{rd}) aspect of the invention—to the same overall goal of preparing a composition comprising activated human CD8⁺ and natural killer (NK) lymphocytes. The method in this aspect comprises the subsequent steps of
a) contacting a first composition of human cells comprising proliferating CD4⁺ lymphocytes with an agent that induces expression of cancer/testis antigens followed by a culturing period that results in said expression of cancer/testis antigens by cells in the first composition; and
b) adding a second composition of human cells comprising unstimulated peripheral blood lymphocytes to the first composition of cells and culturing the combined compositions of cells to stimulate proliferation of CD8⁺ and NK lymphocytes;

wherein a third composition of human cells comprising mature dendritic cells is added in step a or b and at the latest 6 days after initiation of step b, and wherein the first, second and third compositions of human cells are isogeneic.

Thus, this method sets out at the point where a culture of proliferating lymphocytes (the first composition) has been established. Preferably the first composition is enriched for CD4⁺ lymphocytes relative to CD8⁺ lymphocytes, meaning that the ratio CD4⁺ lymphocytes/CD8⁺ lymphocytes is significantly increased compared to that found in normal blood (where the ratio is about 2).

The remaining steps correspond to steps 5-7 in the first aspect of the invention, and any disclosure in relation to these steps in relation to the 1^{st} aspect of the invention relates mutatis mutandis to the 2^{nd} aspect. This is for instance the case with any disclosure that relates to the characteristics of the agent that induces expression of CTAs.

As indicated, the 3 compositions of cells used in the method of the 2^{nd} aspect of the invention are isogeneic, i.e. the cells are derived from cells of the same person or are for other reasons cells having the same genome.

3^{rd} Aspect of the Invention

Again, this aspect has the same overall goal as the methods of the 2 first aspects of the invention, but in this aspect the process sets out after a composition comprising CTA expressing CD4⁺ lymphocytes has been provided. The method thus comprises mixing
a first composition of human cells comprising cancer/testis antigen expressing CD4+ lymphocytes with
a second composition of human cells comprising unstimulated peripheral blood lymphocytes and culturing the combined compositions of cells to stimulate proliferation of CD8+ and NK lymphocytes;

wherein a third composition of human cells comprising mature dendritic cells is added to the combined compositions at the latest 6 days after mixing the first and second composition and wherein the first, second and third compositions of cells are isogeneic.

Also for this method, all considerations provided above relating to process steps and process features apply mutatis mutandis to this aspect of the invention.

### Common Features for Embodiments of all of Aspects 1-3 of the Invention

In all the above aspects of the invention, it is preferred that the mature dendritic cells are unloaded with antigen and that they are non-irradiated. Normally, irradiation of the feeder cells is employed in order to prevent them from proliferating, but the mature dendritic cells used in the present invention do not proliferate or at least they exhibit an acceptably low degree of proliferation. Further, use of peptide loaded dendritic cells has been used to stimulate CD8⁺ cells, but the present methods where the mature DCs are not antigen loaded, has been shown to stimulate proliferation of CD8⁺ cells as well.

In all culturing steps, it has been found that IL-2 advantageously can be applied, cf. the examples.

As already described above, the early addition of the mature DCs as feeder cells is at the latest 6 days after instigation of the co-culture of the CTA expressing CD4⁺ lymphocytes and the non-stimulated lymphocytes; typically this is at the latest 5 days, at the latest 4 days, at the latest 3 days, and at the latest 2 days. Cf. above under the 1^{st} aspect of the invention for details concerning the timing for this early addition. However, it is preferred that the addition of the feeder cells is at the latest or exactly 0, 1 or 2 days after instigation of the co-culture of the CTA expressing CD4⁺ lymphocytes and the non-stimulated lymphocytes.

In all of aspects 1-3 of the invention, the last culture step is typically followed by isolation/recovery of the activated CD8+ and NK lymphocytes. These are then typically subsequently preserved for later use in therapy or they are used directly in the patient from which the cells are derived.

4^{th} Aspect of the Invention

As indicated above, the methods of the 3 first aspects of the invention provide an improved composition of effector cells that are useful in adoptive immune therapy, in particular of the patient from whom the effector cells are originally derived, i.e. where the effector cells are autologous. The 4^{th} aspect hence relates to a method for treatment of cancer in a patient, comprising administering a composition of cells prepared as set forth above for any of aspects 1-3.

A typical treatment regimen provides the patient with at least or exactly 2, at least or exactly 3, or at least of exactly 4 administrations of the effector cells. It is preferred that the patient receives at least 3 treatments. A typical treatment regimen includes 3 administrations of the effector cells with four weeks between each administration.

The administration is normally via the parenteral route, such as the intraveneous, intraarterial route, intra-tumoral route, and intralymphatic route. The cells are conveniently suspended in an aqueous electrolyte-containing medium used for intravenous infusion supplemented with autologous plasma. Good results have been obtained with use of the isotonic infusion liquid Plasmalyte® (Baxter) supplemented with 5% (v/v) autologous serum.

As mentioned above, CTAs are expressed by a wide variety of cancers, so the treatment is against any cancer selected from the group consisting of carcinoma, adenocarcinoma, sarcoma (including liposarcoma, fibrosarcoma, chondrosarcoma, osteosarcoma, leiomyosarcoma, rhabdomyosarcoma), mesothelioma, glioma (in particular glioblastoma), neuroblastoma, medullablastoma, malignant melanoma, neurofibrosarcoma, choriocarcinoma, myeloma, and leukemia. These cancers can be cancer of any tissue origin; of these cancers, the most important are lung cancer, breast cancer, colorectal cancer, colon cancer, stomach cancer, liver cancer, pancreatic cancer, oesophagus cancer, oral cancer, pharyngeal cancer, laryngeal cancer, bronchial cancer, bronchopulmonal cancer, testis cancer, ovarian cancer, cervix cancer, uterus cancer, brain cancer, head and neck cancer, bone cancer, cancer in the skeletal muscles, and cancer of connective tissue.

Related to the 4^{th} aspect of the invention is the 5^{th} aspect, i.e. a composition of cells prepared according to any one of claims 1-15 for use in therapy, in particular for use in a method of the 4^{th} aspect.

6^{th} Aspect of the Invention

Use of mature dendritic cells as feeder cells in a culture of a mixed cell composition initially comprising cancer/testis antigen expressing CD4+ lymphocytes and unstimulated peripheral blood lymphocytes, where the mature dendritic cells are added to the mixed cell composition no later than 6 days after establishment of the mixed cell composition, is considered an invention in its own right. Mature DCs have previously been used as feeder cells, but to the best of the inventors' knowledge not in a composition of CD8⁺ cells blood lymphocytes that are in the process of being activated by CD4⁺ cells.

Another way to put this is that the invention generally provides for the use of mature dendritic cells as feeder cells in a culture of antigen expressing CD4⁺ cells and mixed lymphocytes where the CD4⁺ cells are in the process of activating CD8⁺ cells.

In these uses, it is preferred that the mature dendritic cells and the cells in the mixed cell composition are isogeneic.

### Example 1

Description of the Standard Protocol.

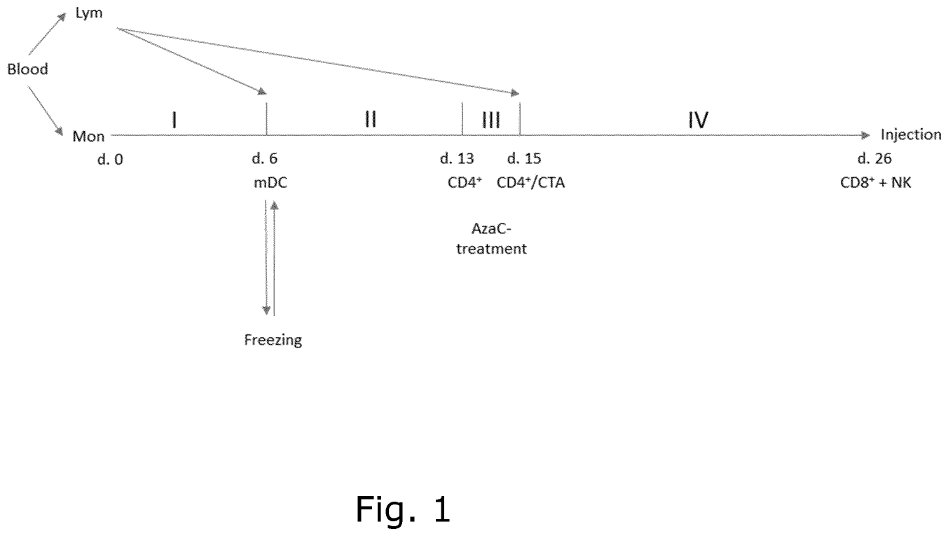

Generation of cytotoxic lymphocytes was achieved according to a variation of the protocol described previously in WO 2008/081035. The entire protocol is outlined in FIG. 1 and set forth in the following:

Day 0

Buffy coats were obtained from the local Blood Bank. Upon arrival, blood (about 60 ml) was diluted with 60 ml of Ca and Mg free Dulbecco's Phosphate Buffered Saline (DPBS, Product No. BE17-512F, Cambrex, Belgium), and approximately 30 ml were layered on 15 ml of Lymphoprep® (Product No. 1053980, AXIS-SHIELD PoC AS, Norway) in four 50 ml tubes.

After the first centrifugation at 200 G, 20 min, 20° C., 15-20 ml of the upper layer of plasma (so-called platelet rich plasma, PRP) were collected to a separate tube, and used for the preparation of serum. For this, CaCl₂ was added to a concentration of 25 mM, and after mixing, the plasma was transferred to a T225 flask (Nunc, Denmark), and placed in a CO₂⁻ incubator. The flask was left in the CO₂-incubator until the next day. Centrifugation of tubes with Lymphoprep® was continued at 460 G, 20 min, 20° C. After termination of centrifugation, mononuclear cells were collected from the interface between Lymphoprep® and plasma to tubes with 25 ml of cold DPBS-EDTA (Cambrex) and washed three times with cold DPBS-EDTA by centrifugation, first at 300 G, then two times at 250 G, each time for 12 min at 4° C. After the last wash, cells were re-suspended in 30 ml of cold Ca and Mg free DPBS, and counted using a Moxi counter. The concentration of monocytes was determined by gating the corresponding peaks of cells. Generation of dendritic cells (DCs) was performed in T225 tissue culture flasks pre-treated with 30 ml of 5% human AB serum in RPMI 1640. After removal of pre-treatment medium, 30 ml of a cell suspension containing 4-5×10⁷ monocytes in AIM-V medium were added. After 30 min of incubation at 37° C., non-adherent lymphocytes were collected, adherent monocytes rinsed twice with pre-warmed RPMI 1640 medium and further cultured in 30 ml of AIM-V medium. The collected lymphocytes were frozen in several aliquots of 25-30×10⁶ cells.

Day 1

GM-CSF and IL-4 (both from Gentaur, Belgium, or CellGenix, Germany) were added to the flask with monocytes to final concentrations of 100 ng/ml and 25 ng/ml, respectively.

The T225 flask with the clotted plasma was transferred to a refrigerator and placed in an inclined position, with the clotted plasma down, and after 15-30 minutes, serum transferred to a 50 ml tube, and transferred to a −20° C. freezer.

Day 2

A tube with the frozen serum was transferred to the refrigerator (4° C.).

Day 3 GM-CSF and IL-4 (both from Gentaur, Belgium, or CellGenix, Germany) were added to the flask with monocytes to final concentrations of 100 ng/ml and 25 ng/ml, respectively.

Tubes with the thawed serum were centrifuged at 2000 G, 15 min, 20° C., and the supernatant was transferred to a new 50 ml tube. This serum (termed “later plasma-derived serum”) was stored at 4° C.

Day 4

IL-18, IL-6, TNF-α (all from Gentaur), and PGE2 (Sigma) were added to final concentrations of 10 ng/ml, 1000 IU/ml, 10 ng/ml and 0.2 μg/ml, respectively, in 10 ml of AIM-V medium.

Day 6

Non-adherent cells were harvested, counted and used for the experiment. For this, the frozen lymphocytes were thawed, counted, and 10⁷ cells were mixed with 10⁶ dendritic cells. After centrifugation, the mixture was re-suspended in 20 ml of lymphocyte medium consisting of AIM-V medium (Gibco, Invitrogen) and 2% autologous plasma derived serum, and placed in a T75 flask. The flask was placed to the side position.

Day 8

IL-2 (Gentaur) was added in 1 ml of AIM-V medium at final concentration of 25 IU/ml.

Day 10

20 ml of fresh lymphocyte medium supplemented with IL-2 (50 U/ml) were added to the flask, and the flask was placed to standard (flat) position.

Day 12 The cell suspension was transferred to a T175 flask, and 40 ml of fresh lymphocyte medium supplemented with IL-2 (50 U/ml) were added to the flask.

Day 13

Cells were harvested, counted, washed, re-suspended in 80 ml of fresh lymphocytes medium to a concentration of 0.5×10⁶/ml, and placed to T175 flask. IL-2 (150 IU/ml) and 10 μM 5-aza-2′-deoxycytidine (obtained from Sigma) were added to the flask.

Day 15

5-Aza-CdR-treated cultures were harvested and counted. 5×10⁶ cells were mixed with 5×10⁶ thawed lymphocytes, and after centrifugation, resuspended in 20 ml of lymphocyte medium, and placed to T75 flask, side position.

Day 17

IL-2 (Gentaur) was added in 1 ml of AIM-V medium to a final concentration of 25 IU/ml.

Day 20

20 ml of fresh lymphocyte medium supplemented with IL-2 (50 U/ml) were added to the flask, and the flask was placed to standard (flat) position.

Day 22

Cell suspension was transferred to T175 flask, and 20 ml of fresh lymphocyte medium supplemented with IL-2 (75 U/ml) were added to the flask.

Day 24

20 ml of fresh lymphocyte medium supplemented with IL-2 (100 U/ml) were added to the flask.

Day 26

Cultures were harvested, counted and used for analysis of phenotype and cytolytic activity.

### Example 2

Effect of Addition of Dendritic Cells in the Beginning of the Immunization Step

In the current invention, an increase in the obtained number of cytotoxic lymphocytes was surprisingly achieved by addition of dendritic cells in the beginning of the “immunization step” (FIG. 1). In the initial experiments, addition of dendritic cells at day 15 (start of the immunization step) was compared to addition at day 17 (two days after initiation of the immunization step, when IL-2 is added to the culture). For this, three cultures were started at day 15 (start of the immunization process, see FIG. 1):

#1 no addition of dendritic cells,

#2 addition of dendritic cells at day 15, and

#3 addition of dendritic cells at day 17.

20 ml of the cultivation medium were dispensed to three T75 TC flasks, placed on the side. The frozen dendritic cells (frozen at day 6 of the whole procedure) were thawed and counted. The amount of 0.5×10⁶ cells was transferred to a new tube, and centrifuged at 300 G, 10 min, 20° C. The pellet was re-suspended with 5 ml of medium taken from the flask #2, and returned back to the flask. 5-Aza-CdR-treated cultures were harvested and counted. 5×10⁶ cells were mixed with 5×10⁶ thawed lymphocytes in each of three centrifuge tubes, and after centrifugation, each pellet was re-suspended in 5 ml of lymphocyte medium taken from one of three T75 flasks, and returned back. The flasks were placed in side position. At day 17, another portion of the frozen dendritic cells (frozen at day 6 of the whole procedure) were thawed and counted. The amount of 0.5×10⁶ cells was transferred to a new tube, and centrifuged at 300 G, 10 min, 20° C. The pellet was re-suspended with 5 ml of medium taken from the flask #3, and returned back to the flask. IL-2 (Gentaur) was added to all flasks in 1 ml of AIM-V medium to a final concentration of 25 IU/ml. All flasks were placed in the side position. Further cultivation was performed as described in Example 1, with exception of more frequent divisions of cultures, which exhibited intensive proliferation. After termination of the total procedure, determination of the total number of generated cells, lytic activity of cells depleted for CD56, and flow cytometric analysis of all cultures were performed.

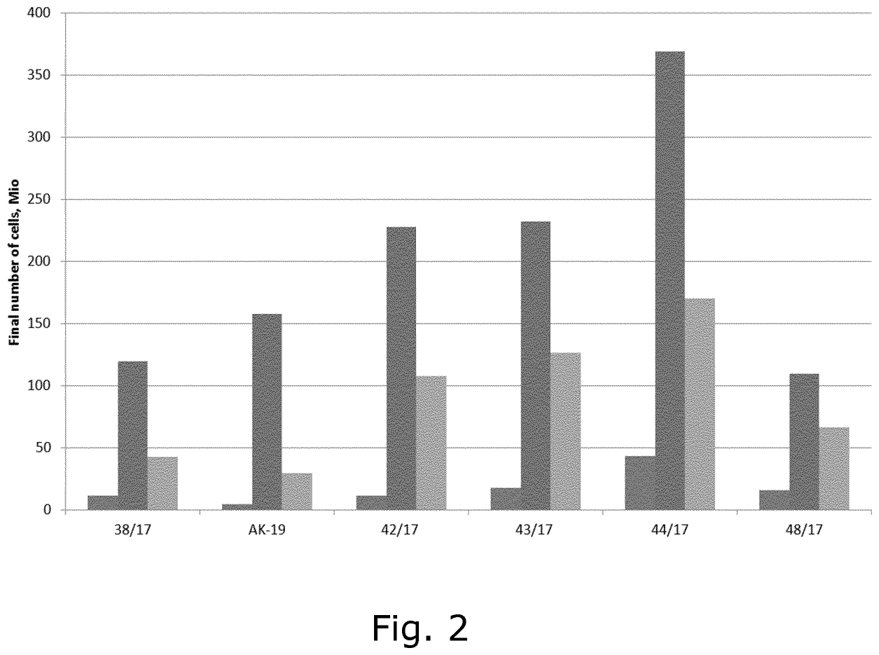

FIG. 2 shows the results of six independent experiments on determination of the total number of cells in control (without addition of dendritic cells) and experimental (with addition of dendritic cells) cultures.

As can be seen, there is a significant increase in the total number of generated cells in all cultures with addition of dendritic cells, with higher effects in cultures with addition at day 15.

To determine the lytic activity of T lymphocytes alone, we performed depletion of CD56+NK cells for several cultures generated from HLA-A2-positive donors using a standard procedure of Miltenyi Biotec, Germany. The cells were washed twice by adding cold DPBS buffer (Cambrex, Denmark, BE02-017F) containing 0.5% Foetal Calf serum (Cambrex, DE14-801F) to the cell suspension. After removal of the buffer the cells were magnetically labelled with CD56 Microbeads (Miltenyi Biotec). The cell suspension was loaded on a LD-column, which was placed in the magnetic field of a MACS Separator. The magnetically labelled CD56-positive cells were retained on the column. The unlabelled cells ran through the column, were collected, and after washing, re-suspended in lymphocyte culture medium. Their lytic activity against the HLA-A2+ breast cancer cell line MDA-MB-231 was determined using real-time cytotoxicity assays with the iCELLigence system (ACEA Biosciences, San Diego, Calif., USA). Tumour cells were seeded at a density of 3×10⁴ cells per well in a total volume of 400 μL of RPMI 1640 medium with 10% FCS. After 2-4 h of initial incubation, 0.2×10⁶ lymphocytes were added in 200 μL of lymphocyte medium. Killing of tumour cells was associated with a decrease in cell impedance (measured as Normalized Cell Index), and was monitored every 15 min for 20-25 h.

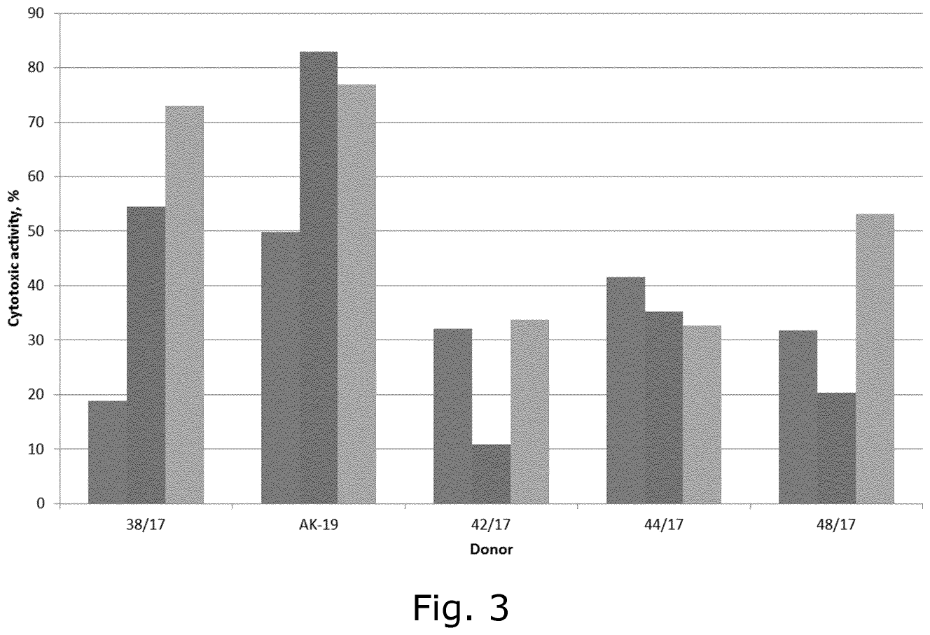

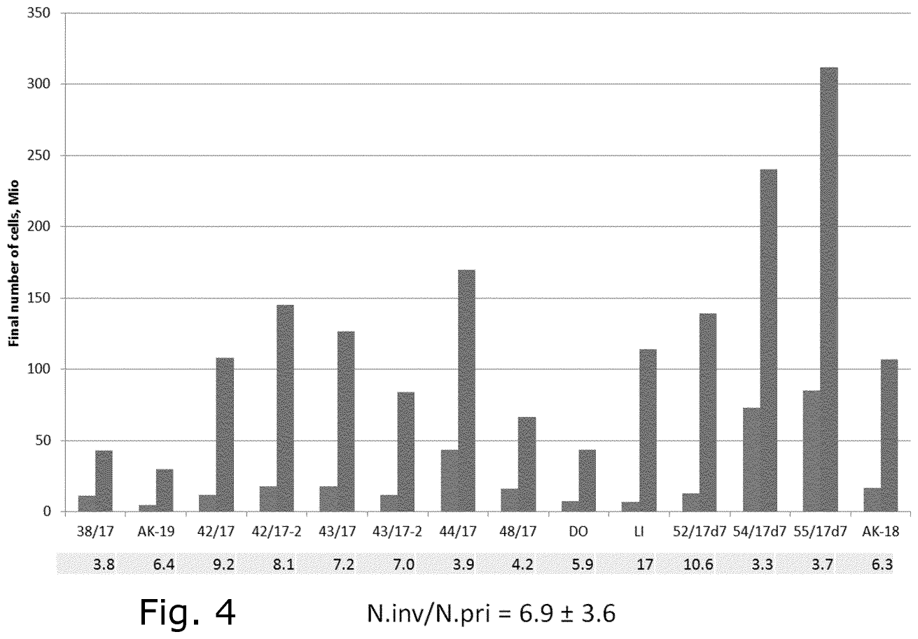

The result of determination of the cytolytic activity of five HLA-A2-positive donors are presented in FIG. 3. As can be seen, addition of dendritic cells at day 17 either has no significant effect on the lytic activity (donors 42/17 and 44/17), or significantly increases the lytic activity (donors 38/17, AK-19 and 48/17). Addition of dendritic cells at day 15 has variable effects, with either increase in activity (donors 38/17 and AK-19), or decrease in activity (donors 42/17 and donors 48/17). Based on these results, we have performed further experiments with addition of dendritic cells at day 17. FIG. 4 summarizes the results of 14 independent experiments on addition of dendritic cells at day 17.

To compare the phenotypes of the generated cells in control and experimental cultures, we performed determination of expression of surface markers by flow cytometric analyses. This was done by staining of cells with the direct conjugated antibodies, CD56-phycoerythrin (PE), CD3-fluorescein isothiocyanate (FITC) and CD62L-phycoerythrin-cyanide 5 (PC5) (BD Biosciences, Denmark, 555516, Beckman Coulter, Sweden, IM1281, IM2655). The recommended isotypic controls were used for the phenotyping of the cells. The cell samples were analyzed using FC500 MPL Flow Cytometer (Beckman Coulter) and the CXP analytical software (Beckman Coulter).

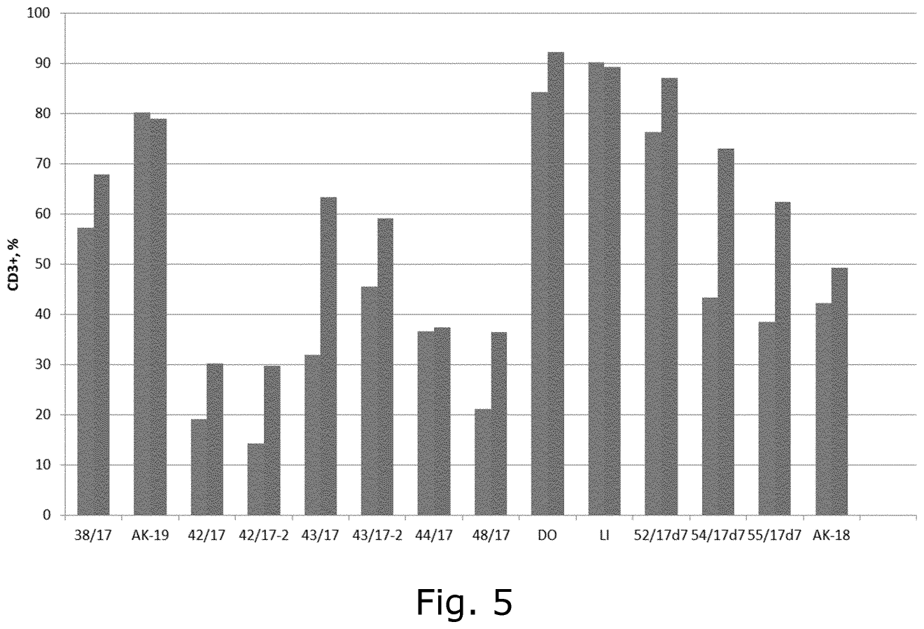

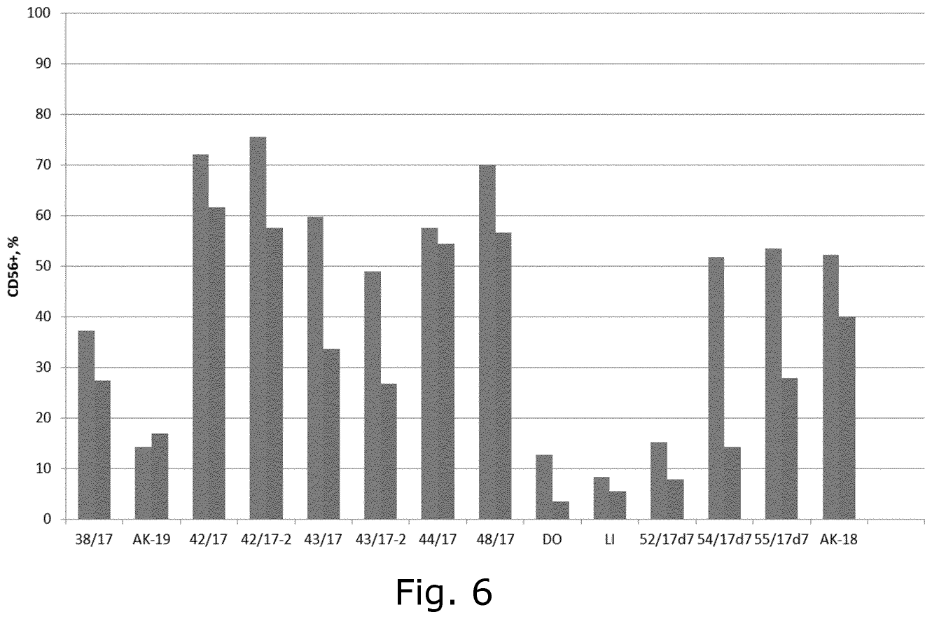

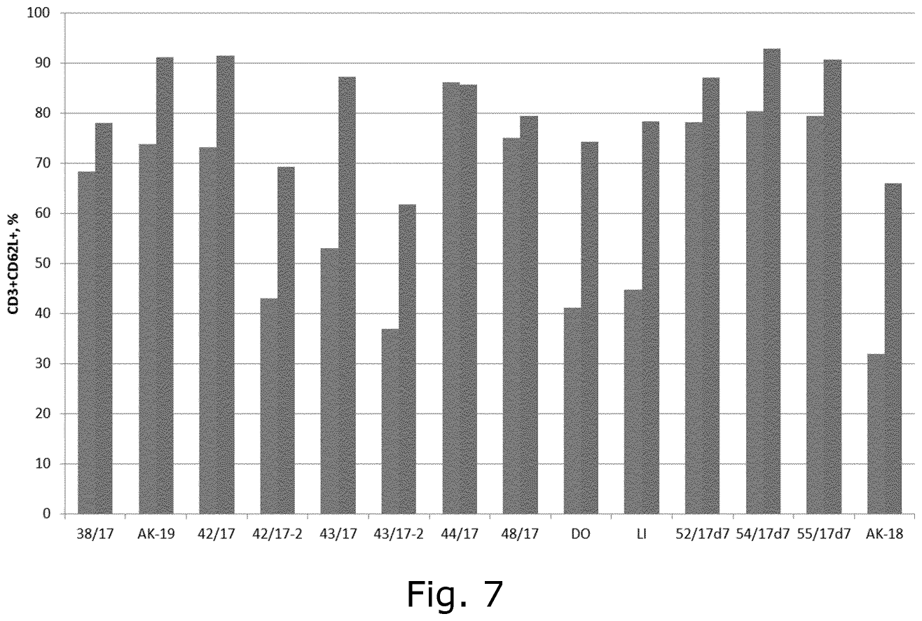

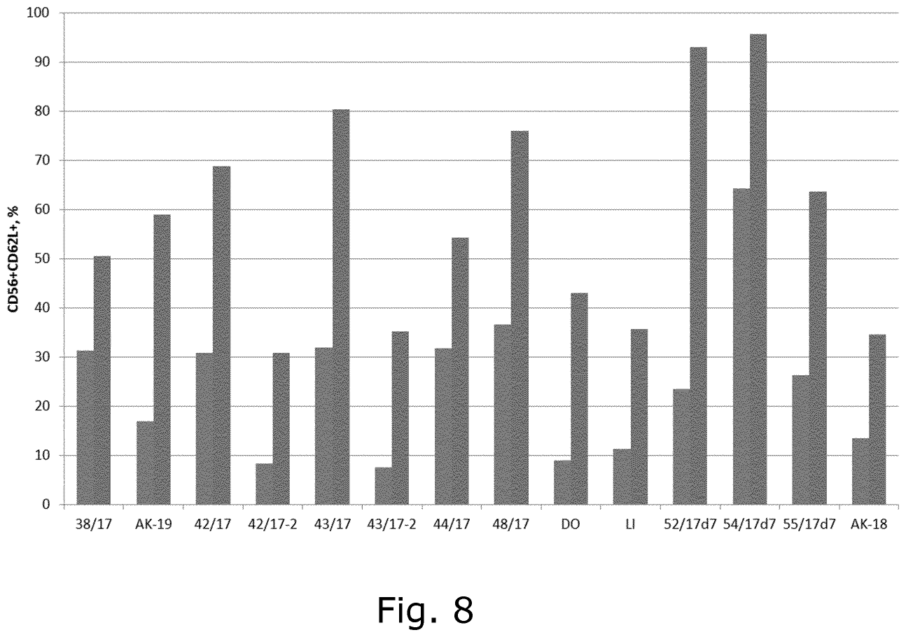

The proliferating cultures where dendritic cells were added at day 17 in general exhibited a higher proportion of T lymphocytes and a lower proportion of NK cells (FIGS. 5 and 6). What is important, expression levels of CD62L is either unchanged, or increased on both T and NK cells (FIGS. 7 and 8). The expression of CD62L is considered to be extremely important for the therapeutic potentials of the injected cells (Gattinoni et al., 2005, Klebanoff et al., 2005).

In summary, an addition of mature dendritic cells at the beginning of the immunisation process lead to a significant increase in total number of generated lymphocytes. Both NK cells and T lymphocytes have the same or higher levels of CD62L, and NK cell-depleted cultures have the same or higher cytolytic activity. All this indicates that this modification of the original procedure leads to generation of effector cells with significantly increased therapeutic potential.

### Example 3

Effect of addition of dendritic cells at day 13 (start of treatment with DNA de-methylating agent 5-aza-2′-deoxycytidine).

In a separate set of experiments we investigated the effect of addition of mature dendritic cells at the start of treatment of CD4-enriched cultures with 5-aza-2′-deoxycytidine (day 13 of the total procedure, see FIG. 1). For this, the frozen dendritic cells (frozen at day 6 of the whole procedure) were thawed and counted. The amount of 2×10⁶ cells was transferred to a new tube, and centrifuged at 300 g, 10 min, 20° C. During centrifugation, 40 ml of lymphocyte medium was placed to each of two T75 flasks. After centrifugation, the pellet was re-suspended with 5 ml of medium taken from one of the flasks, and returned back to this flask.

Proliferating culture of lymphocytes at day 13 was harvested, counted, and 20×10⁶ cells were transferred to each of two centrifuge tubes. After centrifugation, each pellet was re-suspended in 5 ml of medium taken either from the control flask (without dendritic cells), or from the flask with dendritic cells. IL-2 (150 IU/ml) and 10 μM 5-aza-2′-deoxycytidine (Sigma) were added to each flask. Further cultivation of these two cultures was performed as described in Example 1 above.

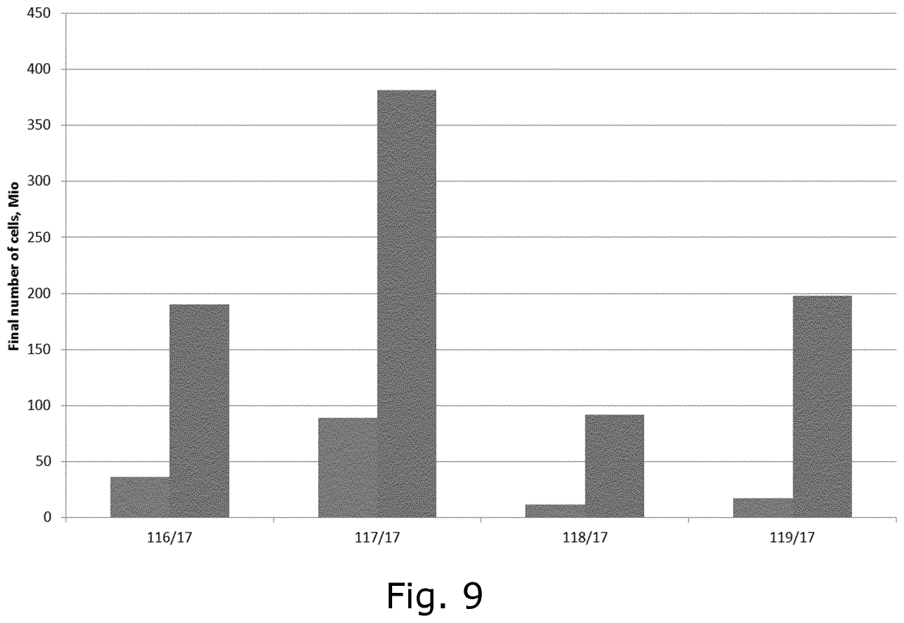

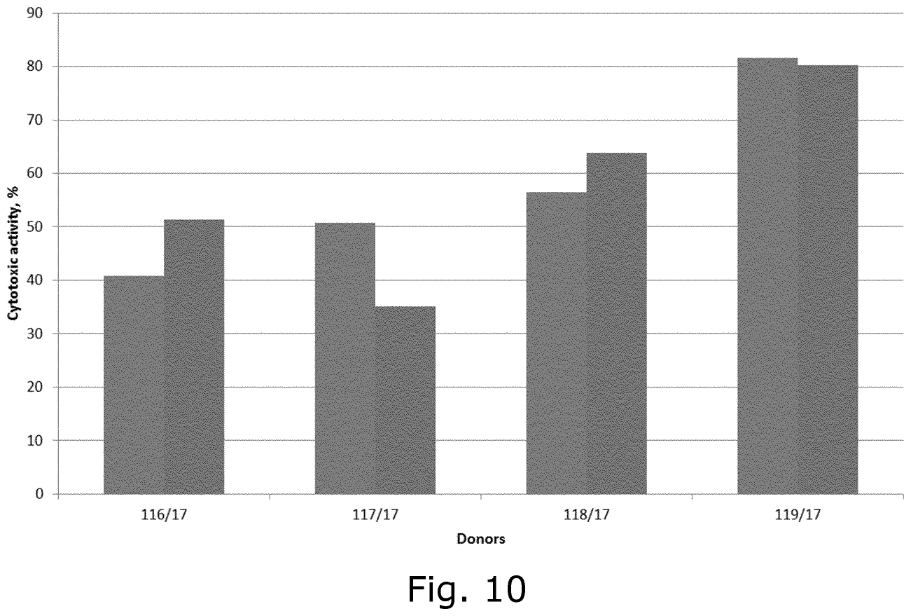

FIG. 9 shows the result of addition of dendritic cells at day 13 on the total number of the generated cells. Here can also be observed the increase in total number of cells, as it was previously demonstrated for addition of dendritic cells at day 15 and 17. Comparison of the lytic activity is presented in FIG. 10. The lytic activities of both cultures are comparable, with exception of donor 117/17, where lower lytic activity was observed in culture with addition of dendritic cells. It is of note that this donor demonstrates a 4.4-fold increase in final number of the generated cells, indicating that the total number of the specific cytotoxic T lymphocytes is increased after addition of dendritic cells.

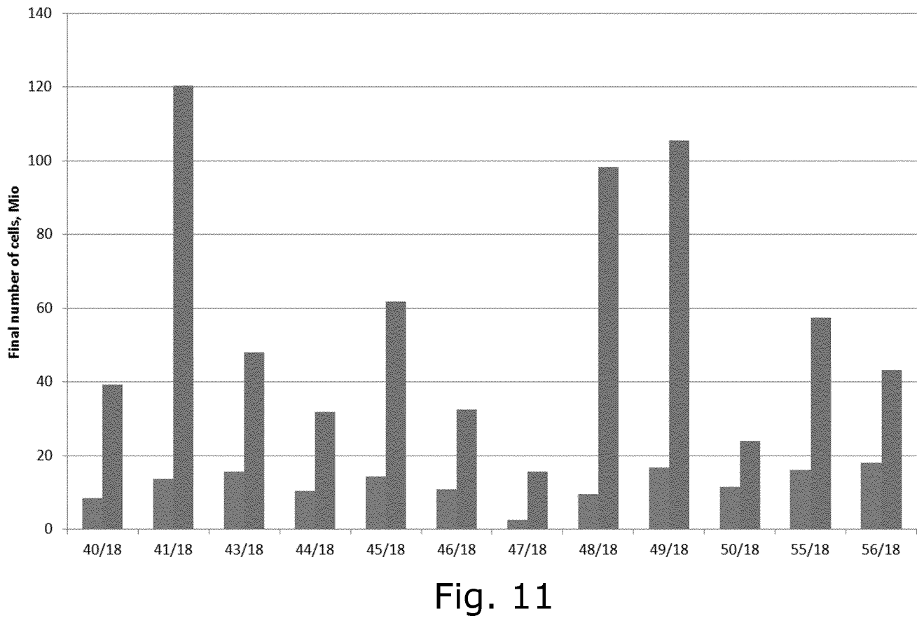

FIG. 11 shows reproducibility of the effect of addition of dendritic cells at day 13 on the final number of cells. The observed average increase is 4.8±2.6, which is slightly lower than what is observed in cultures after addition of dendritic cells at day 17 (6.9±3.6, see FIG. 4).

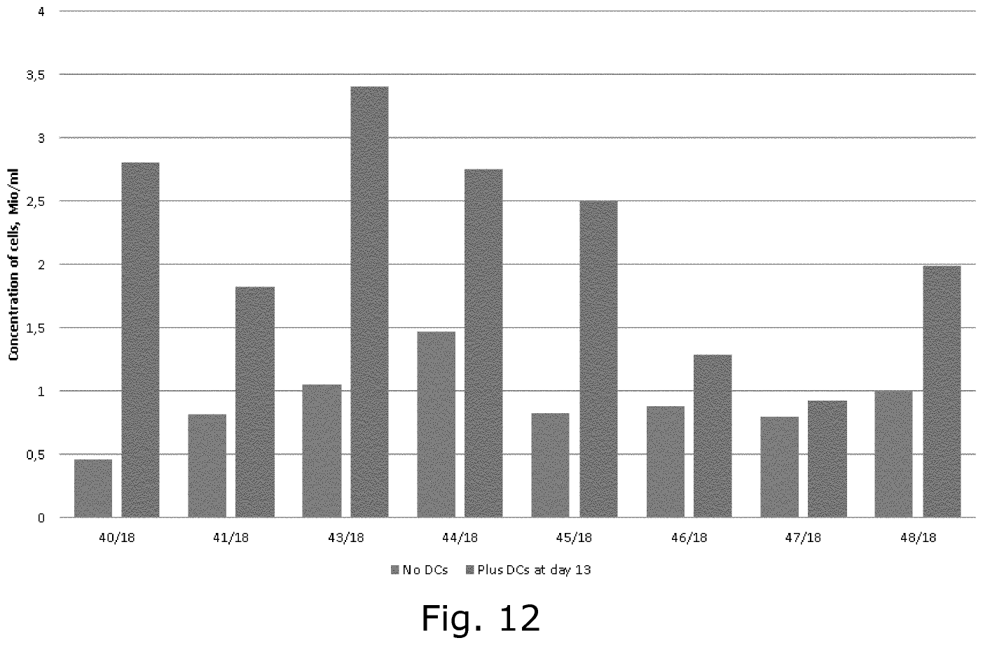

We also investigated the ability of cells in the final product to proliferate in culture in the presence of IL-2. Lymphocytes generated at day 26 were washed and re-suspended in the standard culture medium in the presence of 25 U/ml of interleukin-2 at a concentration of 1×10⁶/ml. 2 ml of cell suspension was placed to wells of 24-well plates. After incubation for 3-4 days, the concentration of cells was determined by cell counting (Moxi cell counter). FIG. 12 shows the results of independent experiments with 8 different donors. Cells from each donor were split in two groups and were cultured with or without addition of dendritic cells. It can be seen that the number of cells in cultures generated with addition of dendritic cells were higher compared to cultures generated in the absence of dendritic cells for all eight independent experiments. This demonstrates that addition of dendritic cells increases the proliferative ability of cells in the final product. Higher proliferative ability of cells prepared in the presence of dendritic cells is believed to reflect higher therapeutic potentials of cells after their injection into the patient.

We also investigated the effect of addition of dendritic cells on final numbers of cells for the selected blood samples taken from glioblastoma patients attending the clinical trial CV006 (randomized, phase II study in patients with newly diagnosed glioblastoma multiforme (EudraCT number: 2015-004058-17, ClinTrials.gov identifier: NCT02799238)).

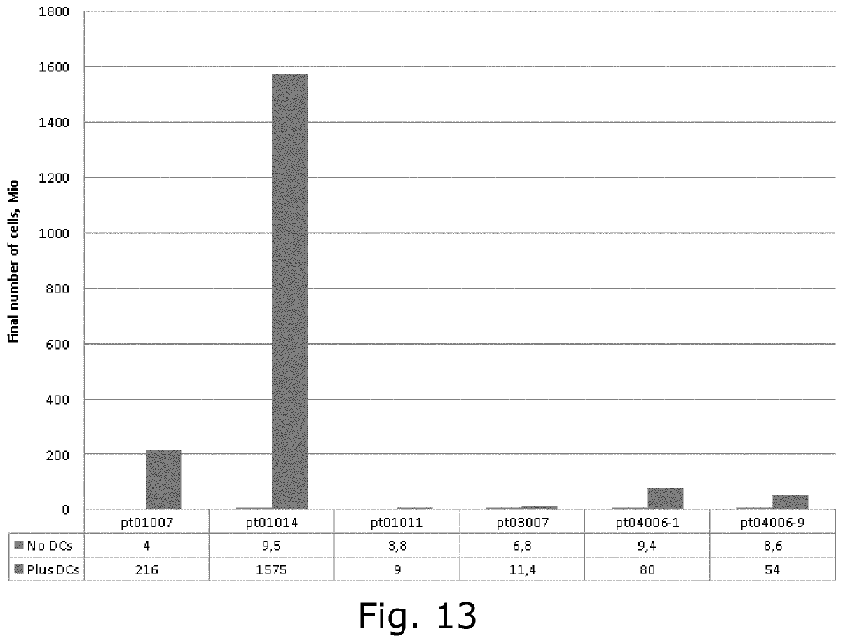

Results presented in FIG. 13 clearly demonstrate the stimulatory effect of addition of dendritic cells, albeit the extent of stimulation is highly variable between different patients. Nevertheless, in all 5 analyses of patient material there was a positive effect of addition of dendritic cells on the proliferation of the lymphocytes. In the bar graph, the cell proliferation observed for cells where no dendritic cells are added during the process is difficult to see but is indicated in tabular form below the bar graph.

In summary, addition of dendritic cells at day 13 also led to an increase in final number of lymphocytes, however with no observed significant increase in lytic activity of T lymphocytes, which has been observed for some cultures where addition of dendritic cells was made at day 17.

### LIST OF REFERENCES

Kirkin A F et al., Nat Commun 2018 Mar. 6; 9(1):785.
Dudley M E et al., J Immunother 2003 July; 26(4):332-42.
Gattinoni L et al., J Clin Invest 2005 June; 115(6):1616-26.
Klebanoff C A et al., Proc Natl Acad Sci USA 2005 Jul. 5; 102(27): 9571-6.

1. The method according to claim 14, comprising, prior to step i),
1) isolating mononuclear cells from a blood sample from a human donor and separating the PBMCs into a fraction enriched for monocytes and a fraction enriched for lymphocytes;
2) culturing a portion of the monocyte-enriched fraction under conditions that facilitate maturation of dendritic cells;
3) subsequently mixing a first portion of the mature dendritic cells obtained in step 2 with a first portion of the lymphocyte fraction obtained in step 1);
4) co-culturing the mixed cells obtained in step 3 to stimulate proliferation of CD4⁺ lymphocytes, thereby increasing the CD4⁺/CD8⁺ ratio compared to the lymphocytes obtained from step 1; and then
5) isolating proliferating lymphocytes from the co-cultured cells of step 4, and subsequently contacting them with an agent that induces expression of cancer/testis antigens followed by a period of culture that results in said expression of cancer/testis antigens; and wherein step i) and ii) comprises;
6) mixing the cancer/testis antigen expressing lymphocytes obtained in step 5 with a second portion of the fraction enriched for lymphocytes in step 1;
7) subsequently culturing the lymphocyte mixture from step 6 to stimulate proliferation of CD8⁺ and NK lymphocytes,
wherein a second portion of mature dendritic cells obtained from step 2 are added to the proliferating lymphocytes in any of steps 5-7 and at the latest 6 days after step 6.

2. The method according to claim 1, wherein steps 1-2 have duration of about 6 days, wherein steps 3-4 have a duration of about 7 days, wherein step 5 has a duration of about 2 days, and wherein steps 6-8 have a duration of about 11 days.

3. The method according to claim 1, wherein the second portion of mature dendritic cells is added in step 5 prior to, concurrently with or after contacting the proliferating lymphocytes with the agent that induces expression of cancer/testis antigens.

4. The method according to claim 1, wherein the second portion of mature dendritic cells is added in step 6 concurrently with or after mixing the cancer/testis antigen expressing lymphocytes with second portion of the fraction enriched for lymphocytes.

5. The method according to claim 1, wherein the second portion of mature dendritic cells is added 13-17 days after commencement of step 2, preferably 15-17 days after commencement of step 2.

6. The method according to claim 1, wherein the first portion of the fraction enriched for lymphocytes is kept frozen between steps 1 and 2, and wherein the second portion of the fraction enriched for lymphocytes is kept frozen between steps 1 and 6.

7. The method according to claim 1, wherein the second portion of the mature dendritic cells is kept frozen between step 2 and its addition in step 5 or 6.

8. The method according to claim 1, wherein step 2 includes addition, during the course of culture, of granulocyte macrophage colony stimulating factor (GM-CSF) as well as Interleukin 4 (IL-4) and/or Interleukin 12 (IL-13), and optionally Interleukin 1b (IL-Ib), Interleukin 6 (IL-6), Tumour Necrosis Factor a (TNF-a), and prostaglandin E2 (PGE2).

9. The method according to claim 14, comprising, prior to step 1) the steps of
a) contacting a first composition of human cells comprising proliferating CD4⁺ lymphocytes with an agent that induces expression of cancer/testis antigens followed by a culturing period that results in said expression of cancer/testis antigens by cells in the first composition; and wherein steps i) and ii) comprise
b) adding a second composition of human cells comprising unstimulated peripheral blood lymphocytes to the first composition of cells and culturing the combined compositions of cells to stimulate proliferation of CD8⁺ and NK lymphocytes;
wherein a third composition of human cells comprising mature dendritic cells is added in step a or b and at the latest 6 days after initiation of step b, and wherein the first, second and third compositions of human cells are isogeneic.

10. The method according to claim 9, wherein the first composition is enriched for CD4⁺ lymphocytes relative to CD8⁺ lymphocytes.

11. The method according to claim 1, wherein the agent that induces expression of cancer/testis antigens is a DNA de-methylating agent or a histone acetylating agent.

12. The method according to claim 11, wherein the DNA de-methylating agent is selected from 5-aza-2′-deoxycytidine (5 Aza-CdR), 5-azacytidine, 5-fluoro-2′-deoxycytidine, guadecitabine, and zebularine, and wherein the histone acetylating agent is Trichostatin A or a depsipeptide.

13. The method according to claim 1, wherein the agent that induces expression of cancer/testis antigens is 5-Aza-CdR.

14. A method for preparation of a composition comprising activated human CD8⁺ and natural killer (NK) lymphocytes, comprising the steps of
i) mixing
a first composition of human cells comprising cancer/testis antigen expressing CD4⁺ lymphocytes with
a second composition of human cells comprising unstimulated peripheral blood lymphocytes and
ii) culturing the combined compositions of cells to stimulate proliferation of CD8⁺ and NK lymphocytes;
wherein a third composition of human cells comprising mature dendritic cells is added to the combined compositions at the latest 6 days after mixing the first and second composition and wherein the first, second and third compositions of cells are isogeneic.

15. The method according to claim 14, wherein the mature dendritic cells are unloaded with antigen and non-irradiated.

16. The method according to claim 14, wherein IL-2 is added during the course of culture of lymphocytes.

17. The method according to claim 14, wherein the time limit of at the latest 6 days is selected from at the latest 5 days, at the latest 4 days, at the latest 3 days, and at the latest 2 days.

18. The method according to claim 17, wherein the at the latest 6 days is selected from 0, 1 and 2 days.

19. The method according to claim 14, which is followed by isolation/recovery of the activated CD8⁺ and NK lymphocytes.

20. A method for treatment of cancer in a patient, comprising administering a composition of cells prepared according to the method of claim 14.

21. The method according to claim 20, wherein the cells are the patient's autologous cells.

22. The method according to claim 20, wherein the patient received at least or exactly 2, at least or exactly 3, or at least of exactly 4 administrations.

23. The method according to any one of claim 20, wherein the administration is via the parenteral route, such as the intraveneous, intraarterial route, intratumoral route, and intralymphatic route.

24. The method according to any one of claim 20, wherein the cancer is selected from the group consisting of carcinoma, adenocarcinoma, sarcoma (including liposarcoma, fibrosarcoma, chondrosarcoma, osteosarcoma, leiomyosarcoma, rhabdomyosarcoma), glioma (in particular glioblastoma), neuroblastoma, medullablastoma, malignant melanoma, neurofibrosarcoma, choriocarcinoma, myeloma, and leukemia.

25-28. (canceled)

29. The method according to claim 9, wherein the agent that induces expression of cancer/testis antigens is a DNA de-methylating agent or a histone acetylating agent.

30. The method according to claim 9 wherein the agent that induces expression of cancer/testis antigens is 5-Aza-CdR.

31. The method according to claim 9, wherein the mature dendritic cells are unloaded with antigen and non-irradiated.

32. The method according to claim 9, wherein IL-2 is added during the course of culture of lymphocytes.

33. The method according to claim 9, wherein the time limit of at the latest 6 days is selected from at the latest 5 days, at the latest 4 days, at the latest 3 days, and at the latest 2 days.

34. The method according to claim 9, which is followed by isolation/recovery of the activated CD8⁺ and NK lymphocytes.

35. The method according to claim 14, wherein the mature dendritic cells are unloaded with antigen and non-irradiated.

36. The method according to claim 14, wherein IL-2 is added during the course of culture of lymphocytes.

37. The method according to claim 14, wherein the time limit of at the latest 6 days is selected from at the latest 5 days, at the latest 4 days, at the latest 3 days, and at the latest 2 days.

38. The method according to claim 14, which is followed by isolation/recovery of the activated CD8⁺ and NK lymphocytes.

39. The method of claim 20, comprising administering a composition of cells prepared according to the method of claim 1.

40. The method of claim 20, comprising administering a composition of cells prepared according to the method of claim 9.
